(19) United States
(12) Patent Application Publication
(10) Pub. No.: US 2013/0052144 A1
(43) Pub. Date: Feb. 28, 2013

(54) DEODORANT FOAMS

(71) Applicant: Henkel AG & Co. KGaA, Duesseldorf (DE)

(72) Inventors: Marcus Claas, Hilden (DE); Bernhard Banowski, Duesseldorf (DE)

(73) Assignee: Henkel AG & Co. KGaA, Duesseldorf (DE)

(21) Appl. No.: 13/662,723

(22) Filed: Oct. 29, 2012

Related U.S. Application Data
(63) Continuation of application No. PCT/EP2011/055194, filed on Apr. 4, 2011.

(30) Foreign Application Priority Data
Apr. 30, 2010 (DE) 10 2010 028 420.3

(51) Int. Cl. A61K 8/34; A61Q 15/00

(52) U.S. Cl. 424/47

(57) ABSTRACT

A body deodorant which is easier to apply, has an improved feeling on the skin and which also leaves no wet feeling includes water, at least one water-soluble polyol selected from the group of polyols having 2-9 C-atoms and 2-6 hydroxyl groups and the polyether polyols having a molecular weight of up to 1000 which are obtained by adding ethylene oxide and/or propylene oxide to said types of polyols, 0.1-10 wt.-% water-soluble surfactant, the composition being packed with an aerosol-propulsion gas in a pressurised container; the propulsion gas being a mixture of propane/butane and dimethylether.

### FIELD OF THE INVENTION

The present invention generally relates to agents for deodorizing the body which are dispensed from pressure vessels and during and shortly after being dispensed exist in the form of a foam.

### BACKGROUND OF THE INVENTION

Deodorants are available on the market in a great number of product formats (sticks, roll-ons, sprays etc.). Sprayable compositions that comprise anti-perspirant active substances are usually formulated in anhydrous form, whereas deodorant formulations are also offered in a water-based form. These aqueous deodorants are then deposited on the skin either as a roll-on or sprayed by means of pump dispensers or propellants.

The application of the water-based products by spraying is considered to be unpleasant by the consumer, because the amounts of highly volatile substances (e.g. alcohols) are too low and the solution requires a relatively long time to dry out in the region of the armpit. The consumer describes a too “wet”, unpleasant feeling. Moreover, as a result of the relatively coarse droplets, the majority of pump atomizers have an unsatisfactory spray pattern that emphasizes the wetting effect. Directly after the application, regular rivulets can be formed on the wetted skin from converging droplets.

Propellant-containing sprays can be sprayed more finely, but do not completely solve the abovementioned problem. Furthermore, the use of propellants (which often make up as much as 85 wt % of the formulation) is not uncontroversial on ecological and economical grounds. The object of the present invention was to provide a solution to the abovementioned problems. It was intended to provide a cost-effective, water-based deodorant formulation that can be applied without the unwanted wetting effect and without high quantities of propellants.

It has now been found that expellable, pleasantly applicable compositions with a rapidly subsiding foam and with little propellant can be provided which are free of the cited disadvantages.

Aqueous deodorant compositions are known for example from WO 96/37184 A2. The compositions disclosed therein have pH values of 3 to 6 and comprise a deodorizing active substance in an aqueous carrier, 10 to 20 wt % of a water-soluble polyol from the group of the polyols having 2 to 9 carbon atoms and 2 to 6 hydroxyl groups and from the polyether polyols with molecular weights of up to 1000, which can be obtained from the addition of ethylene oxide and/or propylene oxide onto such polyols, and 0.1 to 10 wt % of a water-soluble surfactant.

Aqueous cosmetic or pharmaceutical preparations and household products packaged with an aerosol propellant in a pressure vessel equipped with a foam valve are described in DE 4327699 A1. This document proposes the use of alkyl polyglucosides for stable foams.

It is desirable to provide a composition that is applied as a deodorant foam with the help of a propellant mixture. It is further desirable that droplets of the composition do not converge and the unwanted wetting effect does not happen. It is further desirable that the composition is formulated so that clothing is not contaminated either.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An aqueous body deodorant, comprising water; at least one water-soluble polyol from the group of the polyols with 2 to 9 carbon atoms and 2 to 6 hydroxyl groups and the polyether polyols with molecular weights up to 1000, which are obtainable from the addition of ethylene oxide and/or propylene oxide onto such polyols; 0.1 to 10 wt % of a water-soluble surfactant, wherein the composition is packaged with aerosol propellant gas in a pressure vessel and a mixture of propane/butane and dimethyl ether is comprised as the propellant gas.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A subject matter of the invention is an aqueous body deodorant, comprising
water;
at least one water-soluble polyol from the group of the polyols with 2 to 9 carbon atoms and 2 to 6 hydroxyl groups and the polyether polyols with molecular weights up to 1000, which are obtainable from the addition of ethylene oxide and/or propylene oxide onto such polyols;
0.1 to 10 wt % of a water-soluble surfactant,

wherein
the composition is packaged with aerosol propellant gas in a pressure vessel and
a mixture of propane/butane and dimethyl ether is comprised as the propellant gas.

The composition is inventively applied as a deodorant foam with the help of a special propellant mixture. This application requires that the droplets do not converge and the unwanted wetting effect does not happen. Due to the special composition, the foam breaks up very quickly as the armpit is closed, such that the clothing is not contaminated either. Moreover, the foam can be expelled with significantly reduced amounts of propellant.

The compositions according to the invention comprise water that is preferably added in amounts above 30 wt %, relative to the weight of the propellant-containing composition. Particularly preferred aqueous body deodorants according to the invention comprise, relative to the weight of the propellant-containing composition, 25 to 80 wt %, preferably 30 to 75 wt %, more preferably 40 to 70 wt % and in particular 50 to 65 wt % water.

In order to regulate the foam stability, the compositions according to the invention comprise at least one water-soluble polyol from the group of the polyols with 2 to 9 carbon atoms and 2 to 6 hydroxyl groups and the polyether polyols with molecular weights up to 1000, which are obtainable from the addition of ethylene oxide and/or propylene oxide onto such polyols. In this regard, all polyols or polyether polyols are understood to be “water-soluble” when their solubility in water is at least 10 wt % at 20° C. thereby forming clear liquid solutions. Preferred exemplary water-soluble polyols are 1,2-propylene glycol, diethylene glycol, 2-methyl-1,3-propane diol, glycerin, 1,2-butylene glycol, 1,3-butylene glycol, 1,4-butylene glycol, 1,2-pentane diol, 1,5-pentane diol, 1,2-hexane diol, 1,6-hexane diol, 1,2,6-hexane triol, 1,2-octane diol, 1,8-octane diol, dipropylene glycol, tripropylene glycol, diglycerin, triglycerin, polyglycerin, erythritol, sorbitol, methyl glucoside, butyl glucoside, trans-1,4-dimethylolcyclohexane, cis-1,4-dimethylolcyclohexane as well as mixtures of the cited substances. Preferred water-soluble polyether polyols with molecular weights of up to 1000, which are obtainable from the addition of ethylene oxide and/or propylene oxide onto polyols, are selected from PEG-3, PEG-4, PEG-6, PEG-7, PEG-8, PEG-9, PEG-10, PEG-12, PEG-14, PEG-16, PEG-18 and PEG-20 as well as mixtures thereof, wherein PEG-3 to PEG-8 are preferred; also selected from addition products of 10 to 20 mol ethylene oxide on methyl glucoside or butyl glucoside, addition products of ethylene glycol on polypropylene glycols. Preferred inventive deodorant compositions comprise at least one water-soluble polyol with 2 to 9 carbon atoms and 2 to 6 hydroxyl groups which is selected from 1,2-propylene glycol, 2-methyl-1,3-propane diol, glycerin, 1,2-butylene glycol, 1,3-butylene glycol and 1,4-butylene glycol, 1,2-pentane diol, 1,5-pentane diol, 1,2-hexane diol, 1,6-hexane diol, 1,2,6-hexane triol, 1,2-octane diol, 1,8-octane diol, dipropylene glycol, tripropylene glycol, diglycerin, triglycerin, erythritol, sorbitol, as well as mixtures of the abovementioned substances. 1,2-Propylene glycol, 1,2-pentane diol, 1,2-hexane diol, 1,6-hexane diol, 1,2-octane diol, dipropylene glycol, tripropylene glycol as well as mixtures thereof are particularly preferred. The body deodorants according to the invention particularly preferably comprise 1,2-propylene glycol as the water-soluble polyol. Particularly preferred body deodorants according to the invention comprise, relative to the weight of the propellant-containing composition, 1 to 20 wt %, preferably 2.5 to 17.5 wt %, more preferably 4 to 15 wt % and in particular 5 to 12.5 wt % of 1,2-propane diol.

Likewise, in order to regulate the foam stability, the inventive compositions can comprise low amounts of lower alcohols, i.e. monohydric alcohols with 2 or 3 carbon atoms. The addition of ethanol and/or isopropanol has proven particularly valuable because the rate at which the foam breaks up can be effectively controlled in this manner. Particularly preferred body deodorants according to the invention comprise, relative to the weight of the propellant-containing composition, 1 to 20 wt %, preferably 2.5 to 17.5 wt %, more preferably 4 to 15 wt % and in particular 5 to 12.5 wt % ethanol.

The total amount of polyols with 2 to 9 carbon atoms and lower alcohols is preferably relatively low. A particularly attractive foam stability with a particularly low wetting effect is achieved with amounts of less than 25 wt % of the cited alcohols. The body deodorants according to the invention quite particularly preferably comprise, relative to the weight of the propellant-containing composition, a mixture of ethanol and 1,2-propane diol in a total amount of up to 20 wt %, preferably up to 19 wt %, more preferably up to 18 wt % and in particular up to 15 wt %. Further preferred body deodorants according to the invention comprise, relative to the weight of the propellant-containing composition, a mixture of ethanol and 1,2-propane diol in a total amount of up to 20 wt %, preferably up to 19 wt %, more preferably up to 18 wt % and in particular up to 15 wt %, wherein the weight ratio of ethanol to 1,2-propane diol is in the range of 0.7 to 1.3, preferably from 0.9 to 1.1 and is particularly preferably 1. Exceptionally preferred body deodorants according to the invention comprise, each relative to the weight of the propellant-containing composition, 2.5 to 17.5 wt %, preferably 4 to 15 wt % and in particular 5 to 12.5 wt % ethanol as well as 2.5 to 17.5 wt %, preferably 4 to 15 wt % and in particular 5 to 12.5 wt 1,2-propane diol. Further exceptionally preferred body deodorants according to the invention comprise, each relative to the weight of the propellant-containing composition, 2.5 to 17.5 wt %, preferably 4 to 15 wt % and in particular 5 to 12.5 wt % of ethanol as well as 2.5 to 17.5 wt %, preferably 4 to 15 wt % and in particular 5 to 12.5 wt % of 1,2-propane diol, wherein the weight ratio of ethanol to 1,2-propane diol is in the range of 0.7 to 1.3, preferably 0.9 to 1.1 and is particularly preferably 1.

The last named compositions particularly preferably comprise no further lower alcohols and no further polyols with 2 to 9 carbon atoms. Furthermore, exceptionally preferred body deodorants according to the invention comprise, each relative to the weight of the propellant-containing composition, up to 20 wt %, preferably up to 19 wt %, more preferably up to 18 wt % and in particular up to 15 wt % of a mixture of ethanol and 1,2-propane diol and moreover comprise no further monohydric alcohols with 2 or 3 carbon atoms and no further water-soluble polyols with 2 to 9 carbon atoms and no further water-soluble polyether polyols with molecular weights up to 1000, which are obtainable from the addition of ethylene oxide and/or propylene oxide onto such polyols.

The propellant system is of decisive importance for the time-limited foam stability. The inventive compositions comprise a mixture of propane/butane and dimethyl ether. Due to its high hydrophilicity, the dimethyl ether fraction ensures a particularly advantageous, soft foam that breaks up in an optimal manner for the inventive purpose. It is particularly preferred if the weight ratio of dimethyl ether to propane/butane mixture is at least 1, preferably 1.1 to 4, particularly preferably 2 to 3. Particularly preferred body deodorants according to the invention comprise, each relative to the weight of the propellant-containing composition, 1 to 40 wt %, preferably 2.5 to 35 wt %, more preferably 5 to 30 wt % and in particular 10 to 25 wt % of a propellant mixture of propane/butane and dimethyl ether. Preferred compositions comprise, each relative to the weight of the propellant-containing composition, 1 to 20 wt %, preferably 2.5 to 17.5 wt %, more preferably 3 to 15 wt % and in particular 5 to 10 wt % of a propane/butane mixture and 1 to 20 wt %, preferably 2.5 to 17.5 wt %, more preferably 4 to 16 wt % and in particular 5 to 15 wt % of dimethyl ether. Particularly preferred compositions comprise, each relative to the weight of the propellant-containing composition, 3 to 20 wt %, preferably 4 to 17.5 wt %, more preferably 5 to 15 wt % and in particular 5 to 10 wt % of a propane/butane mixture and 3 to 20 wt %, preferably 4 to 17.5 wt %, more preferably 5 to 16 wt % and in particular 10 to 15 wt % of dimethyl ether, wherein the weight ratio of dimethyl ether to propane/butane mixture is at least 1, preferably 1.1 to 4, particularly preferably 2 to 3.

The inventive compositions can comprise, in addition to the obligatory propane/butane/dimethyl ether mixture, 0.5 to 10 wt %, preferably 1-5 wt %, of additional propellants, each relative to the weight of the propellant-containing composition. These additional propellants (propellant gases) are selected from propene, iso-butene, n-pentane, pentene, iso-pentane, iso-pentene, methane, ethane, nitrogen, air, oxygen, nitrous oxide, dichlorofluoromethane, chlorodifluoromethane, chlorofluoromethane, 1,1,2,2-tetrachloro-1-fluoroethane, 1,1,1,2-tetrachloro-2-fluoroethane, 1,2,2-trichloro-1,1-difluoroethane, 1,1,2-trichloro-1,2-difluoroethane, 1,1,1-trichloro-2,2-difluoro-ethane, 2,2-dichloro-1,1,1-trifluoroethane, 1,2-dichloro-1,1,2-trifluoroethane, 2-chloro 1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro-1,1-difluoroethane, 1-chloro-1,2,2-trifluoroethane, 2-chloro-1,1,1-trifluoroethane, 1-chloro-1,1,2-trifluoroethane, 1,2-dichloro-1-fluoroethane, 1,1-dichloro-1-fluoroethane, 2-chloro-1,1-difluoroethane, 1-chloro-1,1-difluoroethane, 1-chloro-2-fluoroethane, 1-chloro-1-fluoroethane, 2-chloro-1,1-difluoroethene, 1,1,1,3-tetrafluoroethane, heptafluoro-n-propane, perfluoroethane, monochlorodifluoromethane, 1,1-difluoroethane, individually or also in combination.

Particularly preferred inventive compositions comprise no additional propellants apart from the obligatory propellants.

Vessels made of metal (aluminum, tin plate, tin), safety or non-splintering plastic or glass come into consideration as the compressed gas vessel, for which the factors pressure and rupture strength, corrosion resistance, facile fillability as well as esthetic considerations, handiness, printability etc. play a role in the choice. Special protective inner lacquers ensure the corrosion resistance against the inventive suspension. An inventively preferred protective inner lacquer is an epoxy-phenol lacquer that is available inter alia under the name Hoba 7407 P. The valves that are used particularly preferably possess an internally lacquered valve disc, wherein lacquer and valve material are compatible with one another. If aluminum valves are used, their valve disc can be internally coated with e.g. Micoflex lacquer. If tin plate valves are inventively employed, their valve discs can be internally coated with e.g. PET (polyethylene terephthalate). The inventive compositions comprise 0.1 to 10 wt %, preferably 0.5 to 5 wt %, particularly preferably 0.8 to 3 wt %, exceptionally preferably 1-2 wt % of at least one water-soluble surfactant. Any surfactant whose water-solubility is at least 1 wt %, preferably at least 5 wt %, in water at 20° C. can be used as the water-soluble surfactant. Inventively preferred surfactants are principally the non-ionic surfactants. Particularly preferred body deodorants according to the invention comprise, relative to the weight of the propellant-containing composition, 0.25 to 7.5 wt %, preferably 0.5 to 5 wt %, more preferably 0.75 to 3 wt % and in particular 0.9 to 1.5 wt % of non-ionic surfactant(s).

Alkyl (oligo)-glucosides of the Formula R¹O(C₆H₁₀O₅)ₙH, in which R¹ is an alkyl group with 8 to 16 carbon atoms, (C₆H₁₀O₅) is a glycoside group and n, the degree of oligomerization, is preferably 1 to 10, are inventively particularly preferred as the non-ionic surfactant. The glycoside group is preferably derived from glucose. As far as the glycoside group is concerned, both monoglycosides, in which a cyclic sugar group is glycosidically linked to the fatty alcohol R¹OH, and also oligomeric glycosides, with a degree of oligomerization of up to 8, are suitable. The average degree of oligomerization is given by the molar fractions of the individual oligomers by dividing the sum of the structural units by the sum of the molecules.

Care should be taken when choosing the surfactant such that the antimicrobial, deodorizing action of the deodorant active substance is not impaired. Although non-ionic surfactants are preferred as a result of their good emulsifying and solubilizing properties, losses in activity, however, can occur with certain antimicrobials. The use of alkyl (oligo)-glycosides in the deodorants according to the invention with a great many deodorizing active substances, e.g. with cationic compounds and with the bis-biguanides (e.g. chlorhexidine gluconate) even leads to a synergistically increased deodorization action that permits such deodorant active substances to be incorporated in particularly low and physiologically better compatible concentrations.

Further surface active substances can also be incorporated, preferably instead of the alkyl glycosides, but also in addition to them; however, the concentration of surfactants should not inventively exceed 10 wt % and their concentration should in fact be as low as possible and only as high as required for retaining the stable emulsification of the deodorant active substances and optionally the fragrances.

Further particularly preferred non-ionic surfactants are addition products of ethylene oxide onto fatty alcohols, i.e. onto linear primary alcohols with 6 to 22 carbon atoms, preferably with 10 to 16 carbon atoms. These addition products of ethylene oxide onto fatty alcohols are preferably comprised in a total amount of 0.1-5 wt %, preferably 0.2 to 1 wt %, particularly preferably 0.5-0.7 wt %, each relative to the weight of the propellant-containing composition. Deceth, Undeceth, Laureth, Trideceth, Myristeth and Ceteth are preferred, each with 8 to 20, preferably with 9 to 15, ethylene oxide units per molecule. Deceth-9, Undeceth-9, Laureth-9, Trideceth-9, Myristeth-9, Ceteth-9, Deceth-10, Undeceth-10, Laureth-10, Trideceth-10, Myristeth-10, Ceteth-10, Deceth-11, Undeceth-11, Laureth-11, Trideceth-11, Myristeth-11, Ceteth-11, Deceth-12, Undeceth-12, Laureth-12, Trideceth-12, Myris-teth-12, Ceteth-12, Deceth-13, Undeceth-13, Laureth-13, Trideceth-13, Myristeth-13, Ceteth-13, Deceth-14, Undeceth-14, Laureth-14, Trideceth-14, Myristeth-14, Ceteth-14, Deceth-15, Undeceth-15, Laureth-15, Trideceth-15, Myristeth-15 and Ceteth-15 are particularly preferred. Trideceth-9 is exceptionally preferred, in particular in a total amount of 0.1-5 wt %, preferably 0.2 to 1 wt %, particularly preferably 0.5-0.7 wt %, each relative to the weight of the propellant-containing composition.

Further inventively particularly preferred non-ionic surfactants are selected from ethoxylated castor oil that may be hydrogenated. An ethoxylated hydrogenated castor oil having 10 to 60 ethylene oxide units per molecule, preferably 20 to 40 ethylene oxide units per molecule, is preferred. An ethoxylated hydrogenated castor oil having 10 to 60 ethylene oxide units per molecule, preferably 20 to 40 ethylene oxide units per molecule, is exceptionally preferred in a total amount of 0.1-5 wt %, preferably 0.2 to 1 wt %, particularly preferably 0.5-0.7 wt %, each relative to the weight of the propellant-containing composition. Mixtures of at least one surfactant, selected from Deceth, Undeceth, Laureth, Trideceth, Myristeth and Ceteth, each with 8 to 20, preferably with 9 to 15 ethylene oxide units per molecule, and ethoxylated hydrogenated castor oil that possesses 10 to 60, preferably 20 to 40, ethylene oxide units per molecule, are particularly preferred. This type of mixture is particularly preferably comprised in a total amount of 0.2-5 wt %, preferably 0.5 to 2 wt %, particularly preferably 1-1.4 wt %, each relative to the weight of the propellant-containing composition.

Further preferred non-ionic surfactants are addition products of ethylene oxide onto fatty acids, in particular onto lauric acid (PEG laurate), palmitic acid (PEG palmitate) and stearic acid (PEG stearate), each preferably with 8 to 20, preferably with 9 to 15 ethylene oxide units per molecule.

Further preferred non-ionic surfactants are fatty acid esters of glycerin ethoxylates as well as addition products of ethylene oxide onto fatty acid partial glycerides, onto sorbitol fatty acid esters or onto methyl glucoside fatty acid esters, each having at least 20 ethylene oxide units per molecule. With fatty acid esters of glycerin ethoxylates as well as addition products of ethylene oxide onto fatty acid partial glycerides, onto sorbitol fatty acid esters or onto methyl glucoside fatty acid esters, each having less than 20 ethylene oxide units per molecule, such as for example PEG-7 glyceryl cocoate, the desired foam properties are not reliably reproducibly obtained.

In order for the pH to be adjusted to 3 to 6, the body deodorants according to the invention comprise a weak acid such as e.g. lactic acid, citric acid, tartaric acid or glycolic acid or a buffer system of such acids and minor amounts of the alkali metal salts of such acids. The pH of the inventive compositions is preferably adjusted to slightly acidic and for particularly preferred body deodorants the pH is 2 to 6, preferably 3 to 5 and in particular 3.5 to 4.5.

The pH is preferably stabilized with a citric acid/citrate buffer. Here, preferred body deodorants according to the invention comprise 0.25-5 wt %, preferably 0.5 to 3.5 wt %, more preferably 0.75 to 3 wt % and in particular 0.9-2 wt %, relative to the weight of the propellant-containing composition. The added deodorizing active substances can, in principle, be any substance known for this purpose and which, as a result of their antimicrobial or esterase inhibiting action, inhibit the microbial perspiration decomposition on the skin. However, astringent substances that counteract the formation of perspiration on the skin can also be used. Exemplary known astringent compounds that are germ inhibiting in low concentrations and are perspiration inhibiting in higher concentrations are the aluminum, zinc and zirconium salts that hydrolyze strongly in an aqueous medium.

Exemplary known esterase inhibiting deodorant active substances are the esters of hydroxycarboxylic acids such as e.g. ethyl lactate or triethyl citrate. Exemplary known antimicrobial deodorant active substances are the salts of benzoic acid, of p-hydroxybenzoic acid, of salicylic acid, of usnic acid or of undecylenic acid. Further suitable deodorant active substances are benzyl alcohol, 2-phenylethanol, phenoxyethanol, farnesol, glycerin monoalkyl-(C8-C16) ethers and diglycerin monoalkyl-(C8-C16) ethers and fatty acid monoglycerides.

For the implementation of the invention, antimicrobial and esterase inhibiting deodorant active substances are preferably comprised in an amount of 0.1 to 15 wt %, particularly preferably 0.4 to 10 wt %, exceptionally preferably 2 to 7 wt %, each relative to the weight of the propellant-containing composition. A preferred group of antimicrobial and deodorizing compounds is that of the quaternary ammonium compounds, the bisbiguanides, e.g. chlorhexidine, and the polybiguanides. The group of the betaines and the cationic phospholipids are particularly preferably suitable for use in the body deodorants according to the invention. Cationic phospholipids of the Formula KPL are particularly suitable

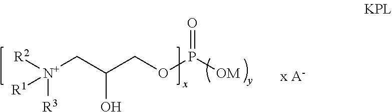

in which R¹ is an alkyl, alkenyl or hydroxyalkyl group with 8 to 22 carbon atoms or an acylaminoalkyl group of the Formula R⁵CONH(CₘH₂ₘ)—, in which R⁵CO is a linear acyl group with 8 to 22 carbon atoms and m=2 or 3, R² and R³ are alkyl groups with 1 to 4 carbon atoms or hydroxyalkyl groups with 2 to 4 carbon atoms or carboxyalkyl groups of the Formula —(CH₂)_{z}—COOM, in which x has a value of 1 to 3 and y a value of (3-x), z has a value of 1 to 3 and M is hydrogen or an alkali metal and A is a water-soluble anion. Preferred body deodorants according to the invention comprise a cationic phospholipid of the Formula KPL as the deodorizing active substance

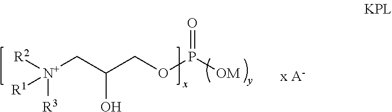

in which R¹ is an alkyl, alkenyl or hydroxyalkyl group with 8 to 22 carbon atoms or an acylaminoalkyl group of the Formula R⁵CONH(CₘH₂O—, in which R⁵CO is a linear acyl group with 8 to 22 carbon atoms and m=2 or 3, R² and R³ are alkyl groups with 1 to 4 carbon atoms or hydroxyalkyl groups with 2 to 4 carbon atoms or carboxyalkyl groups of the Formula —(CH₂)_{z}—COOM, in which x has a value of 1 to 3 and y a value of (3-x), z has a value of 1 to 3 and M is hydrogen or an alkali metal and A is a water-soluble anion. A cationic phospholipid of the Formula KPL with the abovementioned features is preferably comprised in a total amount of 0.1-1 wt %, preferably 0.2 to 0.8 wt %, particularly preferably 0.4-0.6 wt %, each relative to the weight of the propellant-containing composition.

In addition, the inventive compositions can comprise further deodorants. Antimicrobials, antibacterials or germ inhibiting substances, antioxidants or odor adsorbents (e.g. zinc ricinoleate) can be incorporated as the deodorants.

Suitable antimicrobials, antibacterials or germ inhibiting substances are in particular organohalogen compounds as well as organohalides, quaternary ammonium compounds, a series of plant extracts, colloidal, elementary silver, inorganic or organic silver salts, such as in particular silver citrate and silver dihydrogen citrate, and zinc compounds. Halogenated phenol derivatives, such as e.g. hexachlorophene or Irgasan DP 300 (Triclosan, 2,4,4-trichloro-2′-hydroxydiphenyl ether), 3,4,4′-trichlorocarbonilide, Chlorhexidine (1,1′-hexamethylene-bis-[5-(4-chlorophenyl)]-biguanide), Chlorhexidine gluconate, benzalkonium halides and cetylpyridinium chloride, are preferred. Furthermore, sodium bicarbonate, sodium phenol sulfonate and zinc phenol sulfonate as well as e.g. the constituents of linden blossom oil can be incorporated. Weaker acting antimicrobials that, however, have a specific action against gram-positive germs that are responsible for the decomposition of perspiration, can also be incorporated as the deodorant active substance. Benzyl alcohol can also be incorporated as the deodorant active substance. Further antibacterially active deodorants are lantibiotics, glycoglycero lipids, sphingo lipids (ceramides), sterols and other active substances that inhibit the adhesion of bacteria on the skin, e.g. glycosidases, lipases, proteases, carbohydrates, di and oligosaccharide fatty acid esters as well as alkylated mono and oligosaccharides. Preferred deodorant active substances are long chain diols, e.g. 1,2-alkane-(C₅-C₁₈) diols, glycerin mono (C₈-C₁₈) fatty acid esters or, particularly preferably, glycerin mono-(C₆-C₁₆) alkyl ethers, in particular 2-ethylhexyl glycerin ether which are highly compatible with skin and mucous membranes and are active against corynebacteria, as well as in addition phenoxyethanol, phenoxyisopropanol (3-phenoxy-propan-2-ol), aniseed alcohol, 2-methyl-5-phenyl-pentan-1-ol, 1,1-dimethyl-3-phenyl-propan-1-ol, benzyl alcohol, 2-phenylethan-1-ol, 3-phenylpropan-1-ol, 4-phenylbutan-1-ol, 5-phenyl-pentan-1-ol, 2-benzylheptan-1-ol, 2,2-dimethyl-3-phenylpropan-1-ol, 2,2-dimethyl-3-(3′-methyl-phenyl)-propan-1-ol, 2-ethyl-3-phenylpropan-1-ol, 2-ethyl-3-(3′-methylphenyl)-propan-1-ol, 3-(3′-chlorophenyl)-2-ethylpropan-1-ol, 3-(2′-chlorophenyl)-2-ethylpropan-1-ol, 3-(4′-chlorophenyl)-2-ethyl-propan-1-ol, 3-(3′,4′-dichlorophenyl)-2-ethylpropan-1-ol, 2-ethyl-3-(2′-methylphenyl)-propan-1-ol, 2-ethyl-3-(4′-methylphenyl)-propan-1-ol, 3-(3′,4′-dimethylphenyl)-2-ethylpropan-1-ol, 2-ethyl-3-(4′-methoxyphenyl)-propan-1-ol, 3-(3′,4′-dimethoxyphenyl)-2-ethylpropan-1-ol, 2-allyl-3-phenylpropan-1-ol and 2-n-pentyl-3-phenylpropan-1-ol.

Complexants can also support the deodorizing effect, in that they form stable complexes with the oxidative catalytically active heavy metal ions (e.g. iron or copper). Suitable complexants are e.g. the salts of ethylenediaminetetraacetic acid or of nitrilotriacetic acid as well as the salts of 1-hydroxyethane-1,1-diphosphonic acid.

Further preferred inventive compositions comprise at least one fragrance. Suitable fragrances or perfume oils that can be used include individual odoriferous compounds, for example synthetic products of the ester, ether, aldehyde, ketone, alcohol and hydrocarbon type. The phenolic odoriferous compounds include e.g. carvacrol. Odoriferous compounds of the ester type are, for example, benzyl acetate, methyl anthranilate, ortho-t-butylcyclohexyl acetate, p-tert.-butylcyclohexyl acetate, diethyl phthalate, nonane-1,3 diol diacetate, iso-nonyl acetate, iso-nonyl formate, phenylethylphenyl acetate, phenoxyethyl isobutyrate, linalyl acetate, dimethylbenzyl carbinyl acetate, phenylethyl acetate, linalyl benzoate, benzyl formate, ethylmethylphenyl glycinate, allylcyclohexyl propionate, styrallyl propionate, benzyl salicylate, ethyl salicylate, iso-amyl salicylate and 4-nonanolide. The ethers include, for example, benzyl ethyl ether; the aldehydes include, for example, the linear alkanals containing 8 to 18 carbon atoms, citral, citronellal, citronellyloxyacetaldehyde, cyclamen aldehyde, hydroxycitronellal, lilial and bourgeonal; the ketones include, for example, 6-acetyl-1,1,3,4,4,6-hexamethyltetrahydronaphthalene, para-t-amylcyclohexanone, 2-n-heptylcyclopentanone, β-methylnaphthyl ketone and the ionones α-isomethylionone and methyl cedryl ketone; the alcohols include cinnamyl alcohol, anethol, citronellol, dimyrcetol, eugenol, geraniol, linalool, phenylethyl alcohol and terpineol; the hydrocarbons include 1,3,4,6,7,8-hexahydro-4,6,6,7,8,8-hexamethylcyclopenta-a-2-benzopyran, hydroxymethylisopropylcyclopentane, 3-a-methyldodecahydro-6,6,9a-trimethylnaphtho-2(2,1-b)furan, iso-butylquinoline as well as the terpenes and balsams. Mixtures of various fragrances, which together produce an attractive fragrant note, are preferably used.

Suitable perfume oils can also contain natural mixtures of odoriferous substances as are obtainable from vegetal or animal sources, for example pine, citrus, jasmine, lilac, rose or ylang-ylang oil. The ethereal oils of lower volatility that are mostly used as aroma components are also suitable as the perfume oils, e.g. oil of sage, chamomile oil, clove oil, melissa oil, mint oil, cinnamon leaf oil, lime blossom oil, juniper berry oil, vetivert oil, olibanum oil, galbanum oil, laudanum oil, oil of clove buds, iso-eugenol, oil of thyme, rose oil, bergamot oil and geranium oil.

Preferred inventive compositions comprise at least one fragrance in a total amount of 0-10 wt %, preferably 0.2-7 wt %, particularly preferably 0.4 to 6 wt %, exceptionally preferably 1-5 wt %, further exceptionally preferably 2-4 wt %, each relative to the total weight of the propellant-free composition.

Further inventively preferred compositions comprise a content of at least one so-called “skin-cooling active substance”. In the context of the present application, “skin-cooling active substances” are understood to mean active substances that when applied onto the skin generate a pleasant cool feeling as a result of surface anesthetization and stimulation of the nerves that are sensitive to cold, even when the treated skin areas actually have a normal or increased temperature.

Preferred skin-cooling active substances are for example menthol, isopulegol as well menthol derivatives, e.g. menthyl lactate, menthyl pyrrolidone carboxylic acid, menthyl methyl ether, menthoxypropane diol, menthone glycerin acetal (9-methyl-6-(1-methylethyl)-1,4-dioxaspiro(4,5)decane-2-methanol), monomenthyl succinate and 2-hydroxymethyl-3,5,5-trimethylcyclohexanol. Particularly preferred skin-cooling active substances are menthol, isopulegol, menthyl lactate, menthoxypropane diol and menthyl pyrrolidone carboxylic acid.

Preferred inventive compositions comprise at least one skin-cooling active substance in amounts of 0.01-1.5 wt. %; preferably 0.02-0.5 wt. % and particularly preferably 0.05-0.2 wt. %, in each case relative to the total weight of the propellant-free composition.

Apart from fragrances and perfume oils, preferred inventive compositions comprise oils and fats in a total amount of maximum 3 wt %, preferably maximum 2 wt %, particularly preferably maximum 1 wt % and extremely preferably maximum 0.5 wt %, each relative to the total weight of the propellant-containing composition. Too high a content of fats and oils modifies the foaming behavior too strongly and is therefore inventively undesirable.

Another subject matter of the present application is the use of an aqueous body deodorant, comprising
water;
at least one water-soluble polyol from the group of the polyols with 2 to 9 carbon atoms and 2 to 6 hydroxyl groups and the polyether polyols with molecular weights up to 1000, which are obtainable from the addition of ethylene oxide and/or propylene oxide onto such polyols;
0.1 to 10 wt % of a water-soluble surfactant,
a mixture of propane/butane and dimethyl ether as the propellant gas,
   which is packaged with aerosol propellant gas in a pressure vessel, for the non-therapeutic, cosmetic treatment of body odor.

Another subject matter of the present application is a method for the non-therapeutic, cosmetic treatment of body odor, wherein an aqueous body deodorant, comprising
water;
at least one water-soluble polyol from the group of the polyols with 2 to 9 carbon atoms and 2 to 6 hydroxyl groups and the polyether polyols with molecular weights up to 1000, which are obtainable from the addition of ethylene oxide and/or propylene oxide onto such polyols;
0.1 to 10 wt % of a water-soluble surfactant,
a mixture of propane/butane and dimethyl ether as the propellant gas, which is packaged with aerosol propellant gas in a pressure vessel, is applied in an effective amount as a foam onto the skin, in particular the armpit skin.

Preferred inventive compositions, packaged in a pressure vessel (all quantitative data in wt %, relative to the propellant-containing composition)

| | | | | likewise |
|---|---|---|---|---|
| | | particularly | extremely | extremely |
| | preferred | preferred | preferred | preferred |
| Water content | 30-75 | 40-70 | 50-65 | 50-65 |
| Water-soluble | 2.5-17.5 | 4-15 | 5-12.5 | 5-12.5 |
| polyol | | | | |
| according | | | | |
| to claim 1 | | | | |
| Water-soluble | 0.5-5 | 0.8-3 | 1-2 | 1-2 |
| surfactant(s) | | | | |
| Propane/butane | 2.5-17.5 | 3-15 | 5-10 | 5-10 |
| mixture | | | | |
| Dimethyl ether | 2.5-17.5 | 4-16 | 5-15 | 10-15, |
| | | | | wherein the |
| | | | | weight ratio of |
| | | | | dimethyl ether to |
| | | | | propane/butane |
| | | | | mixture is at |
| | | | | least 1, preferably |
| | | | | 1.1 to 4, |
| | | | | particularly |
| | | | | preferably 2 to 3 |

Preferred inventive compositions, packaged in a pressure vessel (all quantitative data in wt %, relative to the propellant-containing composition)

| | | particularly | extremely | likewise extremely |
|---|---|---|---|---|
| | preferred | preferred | preferred | preferred |
| Water content | 30-17.5 | 40-70 | 50-65 | 50-65 |
| 1,2-Propane | 2.5-17.5 | 4-15 | 5-12.5 | 5-12.5 |
| diol | | | | |
| Ethanol | 2.5-17.5 | 4-15 | 5-12.5 | 5-12.5, wherein |
| | | | | the weight ratio of |
| | | | | ethanol to 1,2- |
| | | | | propane diol is 0.7- |
| | | | | 1.3, preferably |
| | | | | 0.9-1.1, |
| | | | | particularly |
| | | | | preferably 1 |
| Non-ionic | 0.5-5 | 0.75-3 | 0.9-1.5 | 0.9-1.5 |
| surfactant(s) | | | | |
| Propane/butane | 2.5-17.5 | 3-15 | 5-10 | 5-10 |
| mixture | | | | |
| Dimethyl ether | 2.5-17.5 | 4-16 | 5-15 | 10-15, wherein |
| | | | | the weight ratio of |
| | | | | dimethyl ether to |
| | | | | propane/butane |
| | | | | mixture is at least |
| | | | | 1, preferably 1.1 to |
| | | | | 4, particularly |
| | | | | preferably 2 to 3. |

Preferred inventive compositions, packaged in a pressure vessel (all quantitative data in wt %, relative to the propellant-containing composition)

| | | | | likewise |
|---|---|---|---|---|
| | | particularly | extremely | extremely |
| | preferred | preferred | preferred | preferred |
| Water content | 30-75 | 40-70 | 50-65 | 50-65 |
| 1,2-Propane | 2.5-17.5 | 4-15 | 5-12.5 | 5-12.5 |
| diol | | | | |
| Ethanol | 2.5-17.5 | 4-15 | 5-12.5 | 5-12.5, |
| | | | | wherein |
| | | | | the weight ratio |
| | | | | of ethanol to 1,2- |
| | | | | propane diol |
| | | | | is 0.7-1.3, |
| | | | | preferably |
| | | | | 0.9-1.1, |
| | | | | particularly |
| | | | | preferably 1 |
| Non-ionic | 0.5-5 | 0.75-3 | 0.9-1.5 | 0.9-1.5 |
| surfactant(s) | | | | |
| Propane/butane | 2.5-17.5 | 3-15 | 5-10 | 5-10 |
| mixture | | | | |
| Dimethyl ether | 2.5-17.5 | 4-16 | 5-15 | 10-15, |
| | | | | wherein |
| | | | | the weight |
| | | | | ratio of |
| | | | | dimethyl ether to |
| | | | | propane/butane |
| | | | | mixture is at least |
| | | | | 1, preferably |
| | | | | 1.1 to 4, |
| | | | | particularly |
| | | | | preferably 2 to 3 |
| pH | 3-5 | 3.5-4.5 | 3.5-4.5 | 3.5-4.5 |
| Deodorant | 0.1-15 | 0.4-10 | 0.4-7 | 0.4-7 |
| active | | | | |
| substance(s) | | | | |
| Cationic | — | 0.1-1 | 0.2-0.8 | 0.4-0.6 |
| phospholipid | | | | |
| of the Formula | | | | |
| KPL according | | | | |
| to claim 8 | | | | |

Preferred inventive compositions, packaged in a pressure vessel (all quantitative data in wt %, relative to the propellant-containing composition)

| | | | | likewise |
|---|---|---|---|---|
| | | particularly | extremely | extremely |
| | preferred | preferred | preferred | preferred |
| Water content | 30-75 | 40-70 | 50-65 | 50-65 |
| 1,2-Propane | 2.5-17.5 | 4-15 | 5-12.5 | 5-12.5 |
| diol | | | | |
| Ethanol | 2.5-17.5 | 4-15 | 5-12.5 | 5-12.5, |
| | | | | wherein |
| | | | | the weight ratio |
| | | | | of ethanol to 1,2- |
| | | | | propane diol |
| | | | | is 0.7-1.3, |
| | | | | preferably |
| | | | | 0.9-1.1, |
| | | | | particularly |
| | | | | preferably 1 |
| Addition | 0.1-5 | 0.2-1 | 0.5-0.7 | 0.5-0.7 |
| product(s) of | | | | |
| ethylene oxide | | | | |
| on fatty alcohol | | | | |
| Propane/butane | 2.5-17.5 | 3-15 | 5-10 | 5-10 |
| mixture | | | | |
| Dimethyl ether | 2.5-17.5 | 4-16 | 5-15 | 10-15, |
| | | | | wherein |
| | | | | the weight ratio |
| | | | | of dimethyl ether |
| | | | | to propane/butane |
| | | | | mixture is at least |
| | | | | 1, preferably |
| | | | | 1.1 to 4, |
| | | | | particularly |
| | | | | preferably 2 to 3 |
| PH | 3-5 | 3.5-4.5 | 3.5-4.5 | 3.5-4.5 |
| Deodorant | 0.1-15 | 0.4-10 | 0.4-7 | 0.4-7 |
| active | | | | |
| substance(s) | | | | |

Preferred inventive compositions, packaged in a pressure vessel (all quantitative data in wt %, relative to the propellant-containing composition)

| | | | | likewise |
|---|---|---|---|---|
| | | particularly | extremely | extremely |
| | preferred | preferred | preferred | preferred |
| Water content | 30-75 | 40-70 | 50-65 | 50-65 |
| 1,2-Propane diol | 2.5-17.5 | 4-15 | 5-12.5 | 5-12.5 |
| Ethanol | 2.5-17.5 | 4-15 | 5-12.5 | 5-12.5, |
| | | | | wherein |
| | | | | the weight |
| | | | | ratio of |
| | | | | ethanol to 1,2- |
| | | | | propane diol |
| | | | | is 0.7-1.3, |
| | | | | preferably |
| | | | | 0.9-1.1, |
| | | | | particularly |
| | | | | preferably 1 |
| Ethoxylated | 0.1-5 | 0.2-1 | 0.5-0.7 | 0.5-0.7 |
| hydrogenated | | | | |
| castor oil that | | | | |
| has 10 to 60 | | | | |
| ethylene | | | | |
| oxide units | | | | |
| per molecule, | | | | |
| preferably | | | | |
| 20 to 40 | | | | |
| ethylene | | | | |
| oxide units | | | | |
| per molecule | | | | |
| Propane/butane | 2.5-17.5 | 3-15 | 5-10 | 5-10 |
| mixture | | | | |
| Dimethyl ether | 2.5-17.5 | 4-16 | 5-15 | 10-15, |
| | | | | wherein |
| | | | | the weight |
| | | | | ratio of |
| | | | | dimethyl |
| | | | | ether to |
| | | | | propane/butane |
| | | | | mixture is |
| | | | | at least 1, |
| | | | | preferably |
| | | | | 1.1 to 4, |
| | | | | particularly |
| | | | | preferably |
| | | | | 2 to 3 |
| pH | 3-5 | 3.5-4.5 | 3.5-4.5 | 3.5-4.5 |
| Deodorant active | 0.1-15 | 0.4-10 | 0.4-7 | 0.4-7 |
| substance(s) | | | | |

referred inventive compositions, packaged in a pressure vessel (all quantitative data in wt %, relative to the propellant-containing composition)

| | | | | likewise |
|---|---|---|---|---|
| | | particularly | extremely | extremely |
| | preferred | preferred | preferred | preferred |
| Water content | 30-75 | 40-70 | 50-65 | 50-65 |
| 1,2-Propane | 2.5-17.5 | 4-15 | 5-12.5 | 5-12.5 |
| diol | | | | |
| Ethanol | 2.5-17.5 | 4-15 | 5-12.5 | 5-12.5, |
| | | | | wherein |
| | | | | the weight |
| | | | | ratio of |
| | | | | ethanol to 1,2- |
| | | | | propane diol |
| | | | | is 0.7-1.3, |
| | | | | preferably |
| | | | | 0.9-1.1, |
| | | | | particularly |
| | | | | preferably 1 |
| Addition | 0.1-5 | 0.2-1 | 0.5-0.7 | 0.5-0.7 wt %, |
| product(s) of | | | | selected from |
| ethylene oxide | | | | Deceth, |
| on fatty | | | | Undeceth, |
| alcohol | | | | Laureth, |
| | | | | Trideceth, |
| | | | | Myristeth and |
| | | | | Ceteth, each |
| | | | | with 8 to 20, |
| | | | | preferably |
| | | | | with 9 to 15 |
| | | | | ethylene oxide |
| | | | | units per |
| | | | | molecule |
| Ethoxylated | 0.1-5 | 0.2-1 | 0.5-0.7 | 0.5-0.7 |
| hydrogenated | | | | |
| castor oil that | | | | |
| has 10 to 60 | | | | |
| ethylene | | | | |
| oxide units | | | | |
| per molecule, | | | | |
| preferably | | | | |
| 20 to 40 | | | | |
| ethylene | | | | |
| oxide units | | | | |
| per molecule | | | | |
| Propane/butane | 2.5-17.5 | 3-15 | 5-10 | 5-10 |
| mixture | | | | |
| Dimethyl ether | 2.5-17.5 | 4-16 | 5-15 | 10-15, |
| | | | | wherein |
| | | | | the weight |
| | | | | ratio of |
| | | | | dimethyl |
| | | | | ether to |
| | | | | propane/butane |
| | | | | mixture is at least |
| | | | | 1, preferably |
| | | | | 1.1 to 4, |
| | | | | particularly |
| | | | | preferably 2 to 3 |
| pH | 3-5 | 3.5-4.5 | 3.5-4.5 | 3.5-4.5 |
| Deodorant | 0.1-15 | 0.4-10 | 0.4-7 | 0.4-7 |
| active | | | | |
| substance(s) | | | | |

Preferred inventive compositions, packaged in a pressure vessel (all quantitative data in wt %, relative to the propellant-containing composition)

| | | | | likewise |
|---|---|---|---|---|
| | | particularly | extremely | extremely |
| | preferred | preferred | preferred | preferred |
| Water content | 30-75 | 40-70 | 50-65 | 50-65 |
| 1,2-Propane | 2.5-17.5 | 4-15 | 5-12.5 | 5-12.5 |
| diol | | | | |
| Ethanol | 2.5-17.5 | 4-15 | 5-12.5 | 5-12.5, |
| | | | | wherein |
| | | | | the weight |
| | | | | ratio of |
| | | | | ethanol to 1,2- |
| | | | | propane diol |
| | | | | is 0.7-1.3, |
| | | | | preferably |
| | | | | 0.9-1.1, |
| | | | | particularly |
| | | | | preferably 1 |
| Addition | 0.1-5 | 0.2-1 | 0.5-0.7 | 0.5-0.7 wt %, |
| product(s) of | | | | selected from |
| ethylene oxide | | | | Deceth, |
| on fatty | | | | Undeceth, |
| alcohol | | | | Laureth, |
| | | | | Trideceth, |
| | | | | Myristeth and |
| | | | | Ceteth, each with |
| | | | | 8 to 20, |
| | | | | preferably |
| | | | | with 9 to 15 |
| | | | | ethylene oxide |
| | | | | units per |
| | | | | molecule |
| Ethoxylated | 0.1-5 | 0.2-1 | 0.5-0.7 | 0.5-0.7 |
| hydrogenated | | | | |
| castor oil | | | | |
| that has | | | | |
| 10 to 60 | | | | |
| ethylene oxide | | | | |
| units per | | | | |
| molecule, | | | | |
| preferably | | | | |
| 20 to 40 | | | | |
| ethylene oxide | | | | |
| units per | | | | |
| molecule | | | | |
| Propane/butane | 2.5-17.5 | 3-15 | 5-10 | 5-10 |
| mixture | | | | |
| Dimethyl ether | 2.5-17.5 | 4-16 | 5-15 | 10-15, |
| | | | | wherein |
| | | | | the weight |
| | | | | ratio of |
| | | | | dimethyl |
| | | | | ether to |
| | | | | propane/butane |
| | | | | mixture is |
| | | | | at least 1, |
| | | | | preferably 1.1 to |
| | | | | 4, particularly |
| | | | | preferably 2 to 3 |
| pH | 3-5 | 3.5-4.5 | 3.5-4.5 | 3.5-4.5 |
| Deodorant | 0.1-15 | 0.4-10 | 0.4-7 | 0.4-7 |
| active | | | | |
| substance(s) | | | | |
| Cationic | — | 0.1-1 | 0.2-0.8 | 0.4-0.6 |
| phospholipid of | | | | |
| the Formula | | | | |
| KPL | | | | |
| according to | | | | |
| claim 8 | | | | |

The following example is intended to illustrate the subject matter of the present application (weight data in wt %).

| | | Active | |
|---|---|---|---|
| | | substance | Aerosol |
| Raw material | INCI | solution | Foam |
| Solubilizer | PEG-40 Hydrogenated | 1.25 | 1 |
| no. 660352 (Symrise) | Castor Oil, Trideceth-9, | | |
| | Propylene Glycol, Aqua | | |
| Perfume | PERFUME | 1 | 0.8 |
| Propane diol-1,2 | PROPYLENE GLYCOL | 7.5 | 6 |
| Ethanol 96% denat. | ALCOHOL DENAT. | 7.5 | 6 |
| Phospholipid Arlasilk | Cocamidopropyl | 0.5 | 0.4 |
| PTC | PGDimonium Chloride | | |
| | Phosphate | | |
| Citric acid | CITRIC ACID | 2 | 1.6 |
| monohydrate | | | |
| Sodium hydroxide (50 wt | Sodium Hydroxide, Aqua | 0.9 | 0.72 |
| %) | (Water) | | |
| Vitamin E Acetate | TOCOPHERYL | 0.05 | 0.04 |
| | ACETATE | | |
| Water, deionized | AQUA (WATER) | 79.3 | 63.44 |
| Propane/butane 15/85 | PROPANE/BUTANE | | 5 |
| (2.1 bar) | | | |
| Dimethyl ether | DIMETHYL ETHER | | 15 |
| Total | | 100.00 | 100.00 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An aqueous body deodorant, comprising
water;
at least one water-soluble polyol selected from the group consisting of polyols with 2 to 9 carbon atoms and 2 to 6 hydroxyl groups and the polyether polyols with molecular weights up to 1000, which are obtainable from the addition of ethylene oxide and/or propylene oxide onto such polyols; and
0.1 to 10 wt % of a water-soluble surfactant,
wherein the composition is a propellant-containing composition that is packaged with a mixture of propane/butane and dimethyl ether as an aerosol propellant gas in a pressure vessel.

2. The body deodorant according to claim 1, comprising, relative to the weight of the propellant-containing composition, 1 to 20 wt % 1,2-propane diol.

3. The body deodorant according to claim 1, comprising, relative to the weight of the propellant-containing composition, 1 to 20 wt % ethanol.

4. The body deodorant according to claim 1, comprising, relative to the weight of the propellant-containing composition, a mixture of ethanol and 1,2-propane diol in a total amount of up to 20 wt %.

5. The body deodorant according to claim 1, comprising, relative to the weight of the propellant-containing composition, 1 to 40 wt % propellant mixture of the propane/butane and the dimethyl ether, wherein preferred compositions comprise 2.5 to 17.5 wt % propane/butane and 2.5 to 17.5 wt % dimethyl ether.

6. The body deodorant according to claim 1, further comprising, relative to the weight of the propellant-containing composition, 0.25 to 7.5 wt % non-ionogenic surfactant(s).

7. The body deodorant according to claim 1, comprising, relative to the weight of the propellant-containing composition, 0.25 to 5 wt % citric acid and/or citrates.

8. The body deodorant according to claim 1, further comprising, as a deodorizing active substance, a cationic phospholipid of the Formula KPL 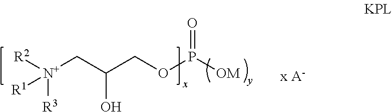
in which R¹ is an alkyl, alkenyl or hydroxyalkyl group with 8 to 22 carbon atoms or an acylaminoalkyl group of the Formula R⁵CONH(CₘH₂ₘ)—, in which R⁵CO is a linear acyl group with 8 to 22 carbon atoms and m=2 or 3, R² and R³ are alkyl groups with 1 to 4 carbon atoms or hydroxyalkyl groups with 2 to 4 carbon atoms or carboxyalkyl groups of the Formula —(CH₂)_{z}—COOM, in which x has a value of 1 to 3 and y a value of (3-x), z has a value of 1 to 3 and M is hydrogen or an alkali metal and A is a water-soluble anion.

9. The body deodorant according to claim 1, comprising, relative to the weight of the propellant-containing composition, 25 to 80 wt % water.

10. The body deodorant according to claim 1, wherein the body deodorant exhibits a pH of 2 to 6.

11. The body deodorant according to claim 1, further comprising, relative to the weight of the propellant-containing composition, 3 to 20 wt % propane/butane mixture and 3 to 20 wt % dimethyl ether, wherein the weight ratio of dimethyl ether to propane/butane mixture is at least 1.

12. The body deodorant according to claim 1, further comprising at least one non-ionogenic surfactant selected from the group consisting of addition products of ethylene oxide onto fatty acids, ethoxylated castor oil that may be hydrogenated, and mixtures thereof.

13. The body deodorant according to claim 1, further comprising a mixture of at least one surfactant, selected from the group consisting of Deceth, Undeceth, Laureth, Trideceth, Myristeth and Ceteth, each with 8 to 20 ethylene oxide units per molecule, and ethoxylated hydrogenated castor oil that possesses 10 to 60 ethylene oxide units per molecule.

14. The body deodorant according to claim 1, comprising, each relative to the weight of the propellant-containing composition, 2.5 to 17.5 wt % ethanol as well as 2.5 to 17.5 wt % 1,2-propane diol, wherein the weight ratio of ethanol to 1,2-propane diol is in the range of 0.7 to 1.3.

15. The body deodorant according to claim 1, wherein, apart from fragrances and perfume oils, oils and fats are comprised in a total amount of maximum 3 wt %, each relative to the total weight of the propellant-containing composition.
